(19) United States
(12) Patent Application Publication
(10) Pub. No.: US 2010/0004391 A1
(43) Pub. Date: Jan. 7, 2010

(54) METHOD FOR DERIVATIZING HAIR WITH A REACTIVE POLYETHYLENE GLYCOL

(76) Inventors: David Mark Haddleton, Coventry (GB); Ezat Khoshdel, Wirral (GB); Julien Nicolas, Paris (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/515,016

(22) PCT Filed: Nov. 14, 2007
(86) PCT No.: PCT/EP2007/062325

§ 371 Date: Sep. 3, 2009

(30) Foreign Application Priority Data
Nov. 17, 2006 (EP) 06124294.7

(51) Int. Cl. A61K 8/86; A61K 8/81; A61Q 5/12; C08G 63/91

(52) U.S. Cl. 525/54.1; 514/723; 424/70.16; 530/357

(57) ABSTRACT

A method of protecting hair by covalently bonding to the hair a polymeric compound comprising polyethylene glycol.

### FIELD OF THE INVENTION

The invention relates a method of protecting the hair fibre and other keratineous materials from harsh environmental conditions and treatments. The method is particularly suitable for the treatment of hair which is dry, damaged and/or prone to manageability problems.

### BACKGROUND AND PRIOR ART

Hair can suffer damage from a number of sources. For example, environmental sources of hair damage include such as exposure to UV and chlorine. Chemical sources of hair damage include treatments such as bleaching, perming and straightening, and overly frequent washing with harsh surfactant-based cleansing shampoo compositions. Mechanical sources of hair damage include excessive brushing and combing and prolonged use of heated appliances for drying and styling the hair.

Damage to the hair typically manifests itself in cuticle and protein loss from the hair fibre, hair fibre dryness, hair fibre brittleness, hair breakage and frayed or split ends. Dry, damaged hair is particularly prone to manageability problems, resulting in symptoms such as “flyaway” hair which is difficult to style or which does not retain a style, especially under conditions such as high humidity.

Various organic molecules and combinations thereof have been suggested for use in the treatment of dry, damaged and/or unmanageable hair.

WO 2004054526 describes hair treatment compositions for the care and repair of damaged hair, and for improving hair manageability, comprising a disaccharide, (in particular trehalose), and a diacid (in particular adipic acid).

WO 2004054525 describes hair treatment compositions for the care and repair of damaged hair, and for improving hair manageability, comprising a disaccharide (in particular trehalose), and a diol (in particular 3-methyl-1,3-butanediol).

WO 2004006874 describes hair treatment compositions for repairing and preventing the principal symptoms of damaged hair, comprising specific branched amine and/or hydroxy compounds (in particular 3,3-dimethyl-1,2-butanediol).

All the above modifications deal with influencing the hair fibre properties by non-covalent interactions such as hydrogen bonding and electrostatic and hydrophobic interactions. The benefits provided by such technologies is not permanent as the benefit agents can easily be washed off. Therefore there is a need to find solutions that are more permanent and long lasting. The present invention addresses such a need. Thus the present invention relates to a method to shield the hair from harsh treatments and external atmospheric conditions, the method also provides a moisturisation benefit.

### SUMMARY OF THE INVENTION

The present provides a method of protecting hair by covalently bonding to the hair a polymeric compound comprising polyethylene glycol.

The invention also provides the use of an reactive derivative of polyethylene glycol for derivatising hair.

A further aspect of the invention is a hair fibre covalently bonded to a polymeric compound comprising polyethylene glycol.

### DETAILED DESCRIPTION

The present invention relates to a method of protecting hair by covalently bonding to the hair a polymeric compound comprising polyethylene glycol. Modification of proteins with polyethyleneglycole is commonly known as PEGylation.

It is preferred if the polymeric compound comprises at least one polymeric segment of polyethylene glycol covalently attached to a central backbone, preferably the central backbone comprises a acrylate, methacrylate group or mixtures thereof. Thus one way of describing a preferred polymeric compound comprising polyethylene glycol, is of a central backbone with a cluster of PEG polymers or PEG segments.

It is preferred if the molecular weight Mw of the polyethylene glycol or polyethylene glycol segment is from 1,000 to 100,000 more preferably from 5,000 to 60,000.

It is also preferable if the number of acrylate, methacrylate groups within the central back bone is from 1 to 20, preferably from 2 to 10.

In addition to the above the polymeric compound comprising polyethylene glycol may comprise other polymeric units.

PEGylation by water soluble polymers and predominately α-functional poly(ethylene glycol) (PEG), can be introduced into proteins by a number of strategies, for example those described in Duncan, R. Nat. Rev. Drug Discov. 2003, 2, 347; Harris, J. M.; Chess, R. B. Nat. Rev. Drug Discov. 2003, 2, 214; Roberts, M. J.; Bentley, M. D.; Harris, J. M. Adv. Drug Deliver. Rev. 2002, 54, 459.

The hair can be PEGylated using the reactive derivative of polyethylene glycol especially preferred are the acrylated polyethylene glycol reactive or methacrylated polyethylene polyethylene glycol or mixtures thereof. The preferred reactive group on the reactive derivative of polyethylene glycol is selected from the group consisting of is α-aldehyde, α-maleimide, α-N-hydroxy succinimide, or α-azlactone.

Preferred methods for PEGylation are described in the following documents:

a)—Aldehyde Terminallyα Functional Methacrylic Polymers from Living Radical Polymerization: Application in Protein Conjugation “Pegylation” Lei Tao, Giuseppe Mantovani, Francois Lecolley, and David M. Haddleton, J. AM. CHEM. SOC. 2004, 126, 13220.
b) A new approach to bioconjugates for proteins and peptides (“pegylation”) utilising living radical polymerisation, Franc ois Lecolley, Lei Tao, Giuseppe Mantovani, Ian Durkin, Sylvie Lautru and David M. Haddleton, Chem. Commun., 2004, 2026. and
c) Design and Synthesis of N-Maleimido-Functionalized Hydrophilic Polymers via Copper-Mediated Living Radical Polymerization: A Suitable Alternative to PEGylation Chemistry, Giuseppe Mantovani, Francüois Lecolley, Lei Tao, David M. Haddleton, Joost Clerx, Jeroen J. L. M. Cornelissen, and Kelly Velonia, J. AM. CHEM. SOC. 2005, 127, 2966-2973.

A particularly preferred method of PEGylation is when the reactive group is α-N-hydroxy succinimide such as the method of (b) described above i.e. exploiting the reaction of an activated NHS-ester chain-end polymer with the amine groups (present in lysine) of the hair fibre. This strategy is very robust and introduces a cluster of hydrophilic groups such as polyethyleneglycols.

The level of polymeric compound comprising polyethylene glycol is preferably from 0.01 to 15 wt. % of the total composition, more preferably from 0.1 to 10 wt. %.

### Product Form

The final product form of hair treatment compositions according to the invention may suitably be, for example, shampoos, conditioners, sprays, mousses, gels, waxes or lotions.

Preferred product forms rinse off products, particularly shampoos and post-wash conditioners

The pH of the formulations of the invention are in the range from pH 3 to pH 11, more preferably used at a pH from 3 to 8.

### Hair Treatment Composition Base Formulation

The composition of the invention may be a shampoo formulation. Shampoo compositions preferably comprise one or more cleansing surfactants, which are cosmetically acceptable and suitable for topical application to the hair. Further surfactants may be present as emulsifiers.

Suitable cleansing surfactants, are selected from anionic, amphoteric and zwitterionic surfactants, and mixtures thereof. The cleansing surfactant may be the same surfactant as the emulsifier, or may be different.

### Anionic Cleansing Surfactant

Shampoo compositions according to the invention will typically comprise one or more anionic cleansing surfactants which are cosmetically acceptable and suitable for topical application to the hair.

Examples of suitable anionic cleansing surfactants are the alkyl sulphates, alkyl ether sulphates, alkaryl sulphonates, alkanoyl isethionates, alkyl succinates, alkyl sulphosuccinates, N-alkyl sarcosinates, alkyl phosphates, alkyl ether phosphates, alkyl ether carboxylates, and alpha-olefin sulphonates, especially their sodium, magnesium, ammonium and mono-, di- and triethanolamine salts. The alkyl and acyl groups generally contain from 8 to 18 carbon atoms and may be unsaturated. The alkyl ether sulphates, alkyl ether phosphates and alkyl ether carboxylates may contain from 1 to 10 ethylene oxide or propylene oxide units per molecule.

Typical anionic cleansing surfactants for use in shampoo compositions of the invention include sodium oleyl sulpho succinate, ammonium lauryl sulphosuccinate, ammonium lauryl sulphate, sodium cocoyl isethionate, sodium lauryl isethionate and sodium N-lauryl sarcosinate. The most preferred anionic surfactants are sodium lauryl sulphate, sodium lauryl ether sulphate(n)EO, (where n ranges from 1 to 3), ammonium lauryl sulphate and ammonium lauryl ether sulphate(n)EO, (where n ranges from 1 to 3).

The total amount of anionic cleansing surfactant in shampoo compositions of the invention is generally from 5 to 30, preferably from 6 to 20, more preferably from 8 to 16 wt % of the total composition.

### Co-Surfactant

The shampoo composition can optionally include co-surfactants, preferably an amphoteric or zwitterionic surfactant, which can be included in an amount ranging from 0 to about 8, preferably from 1 to 4 wt %.

Examples of amphoteric and zwitterionic surfactants include, alkyl betaines, alkyl amidopropyl betaines, alkyl sulphobetaines (sultaines), alkyl glycinates, alkyl carboxyglycinates, alkyl amphopropionates, alkylamphoglycinates, alkyl amidopropyl hydroxysultaines, acyl taurates and acyl glutamates, wherein the alkyl and acyl groups have from 8 to 19 carbon atoms. Typical amphoteric and zwitterionic surfactants for use in shampoos of the invention include lauryl amine oxide, cocodimethyl sulphopropyl betaine and preferably lauryl betaine, cocamidopropyl betaine and sodium cocamphopropionate.

Another preferred co-surfactant is a nonionic surfactant, which can be included in an amount ranging from 0 to 8 wt %, preferably from 2 to 5 wt % of the total composition.

For example, representative nonionic surfactants that can be included in shampoo compositions of the invention include condensation products of aliphatic (C₈-C₁₈) primary or secondary linear or branched chain alcohols or phenols with alkylene oxides, usually ethylene oxide and generally having from 6 to 30 ethylene oxide groups.

Further nonionic surfactants which can be included in shampoo compositions of the invention are the alkyl polyglycosides (APGs). Typically, the APG is one which comprises an alkyl group connected (optionally via a bridging group) to a block of one or more glycosyl groups. Preferred APGs are defined by the following formula:

RO-(G)ₙ

wherein R is a branched or straight chain C₅ to C₂₀ alkyl or alkenyl group, G is a saccharide group and n is from 1 to 10.

The shampoo composition can also optionally include one or more cationic co-surfactants included in an amount ranging from 0.01 to 10, more preferably from 0.05 to 5, most preferably from 0.05 to 2 wt % of the total composition. Useful cationic surfactants are described herein in relation to conditioner compositions.

The total amount of surfactant (including any co-surfactant, and/or any emulsifier) in shampoo compositions of the invention is generally from 5 to 50, preferably from 5 to 30, more preferably from 10 to 25 wt %.

### Cationic Deposition Polymer

A cationic polymer is a preferred ingredient, especially in shampoo compositions of the invention.

The cationic polymer may be a homopolymer or be formed from two or more types of monomers. The molecular weight of the polymer will generally be between 5 000 and 10 000 000 Dalton, typically at least 10 000 and preferably from 100 000 to 2 000 000. The polymers will have cationic nitrogen containing groups such as quaternary ammonium or protonated amino groups, or a mixture thereof.

The cationic nitrogen-containing group will generally be present as a substituent on a fraction of the total monomer units of the cationic polymer. Thus when the polymer is not a homopolymer it can contain spacer non-cationic monomer units. Such polymers are described in the CTFA Cosmetic Ingredient Directory, 3rd edition. The ratio of the cationic to non-cationic monomer units is selected to give a polymer having a cationic charge density in the required range.

The cationic deposition polymer will generally be present in compositions of the invention at levels of from 0.01 to 5, preferably from 0.02 to 1, more preferably from 0.04 to 0.5 percent by weight of the composition.

### Conditioning Surfactant

Conditioner compositions usually comprise one or more conditioning surfactants which are cosmetically acceptable and suitable for topical application to the hair.

Suitable conditioning surfactants are selected from cationic surfactants, used singly or in a mixture.

Cationic surfactants useful in compositions of the invention contain amino or quaternary ammonium hydrophilic moieties which are positively charged when dissolved in the aqueous composition of the present invention.

Examples of suitable cationic surfactants are those corresponding to the general formula:

[N(R₁)(R₂)(R₃)(R₄)]⁺(X)⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

### Fatty Materials

Conditioner compositions of the invention preferably additionally comprise fatty materials. The combined use of fatty materials and cationic surfactants in conditioning compositions is believed to be especially advantageous, because this leads to the formation of a structured phase, in which the cationic surfactant is dispersed.

By “fatty material” is meant a fatty alcohol, an alkoxylated fatty alcohol, a fatty acid or a mixture thereof.

Preferably, the alkyl chain of the fatty material is fully saturated.

Representative fatty materials comprise from 8 to 22 carbon atoms, more preferably 16 to 22. Examples of suitable fatty alcohols include cetyl alcohol, stearyl alcohol and mixtures thereof. The use of these materials is also advantageous in that they contribute to the overall conditioning properties of compositions of the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty alcohol material in conditioners of the invention is suitably from 0.01 to 15 wt %, preferably from 0.1 to 10 wt % of the total composition. The weight ratio of cationic surfactant to fatty alcohol is suitably from 10:1 to 1:10, preferably from 4:1 to 1:8, optimally from 1:1 to 1:7.

### Suspending Agents

In a preferred embodiment, the shampoo compositions of this invention further comprises from 0.1 to 5 wt % of a suspending agent for the coated particles. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives. Polyacrylic acid is available commercially as Carbopol 420, Carbopol 488 or Carbopol 493. Polymers of acrylic acid cross-linked with a polyfunctional agent may also be used, they are available commercially as Carbopol 910, Carbopol 934, Carbopol 940, Carbopol 941 and Carbopol 980. An example of a suitable copolymer of a carboxylic acid containing a monomer and acrylic acid esters is Carbopol 1342. All Carbopol (trade mark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

The suspending agent is preferably a polymeric suspending agent.

### Styling Polymers

If the composition is a styling product it is preferred if a styling polymer is present

The hair styling polymer if present is preferably present in the compositions of the invention in an amount of from 0.001% to 10% by weight, more preferably from 0.1% to 10% by weight, such as from 1% to 8% by weight.

Hair styling polymers are well known. Suitable hair styling polymers include commercially available polymers that contain moieties that render the polymers cationic, anionic, amphoteric or nonionic in nature. Suitable hair styling polymers include, for example, block and graft copolymers. The polymers may be synthetic or naturally derived.

The amount of the polymer may range from 0.5 to 10%, preferably 0.75 to 6% by weight based on total weight of the composition.

### Adjuvants

The compositions of the present invention may also contain adjuvants suitable for hair care. Generally such ingredients are included individually at a level of up to 2, preferably up to 1 wt % of the total composition.

Suitable hair care adjuvants, include amino acids and ceramides.

The invention will now be illustrated by the following non-limiting Examples

### EXAMPLES

### Hair Samples

European hair fibres were bleached two times with L'Oreal platifiz precision powder and Oxydant crème (1:1.5) for 30 minutes, rinsed completely with running water from the tap, and naturally dried overnight.

Preparation of Reactive NHS-PEG-Functionalised Polymers with Fluorescent Marker

### Materials

Copper(I) bromide (Cu(I) Br, Aldrich, 98%) was purified according to the method of Keller and Wycoff (Keller, R. N.; Wycoff, H. D. Inorg. Synth. 1946, 1-4). N-(ethyl)-2-pyridylmethanimine was prepared as described earlier and stored at 0° C. under inert atmosphere (Haddleton, D. M.; Crossman, M. C.; Dana, B. H.; Duncalf, D. J.; Heming, A. M.; Kukulj, D.; Shooter, A. J. Macromolecules 1999, 32, 2110-2119). Diethylene glycol dimethyl ether (Aldrich, 99.5%), triethylamine (TEA, VWR, >99%), poly(ethylene glycol) methyl ether methacrylate (PEGMA₄₇₅, Mₙ=475 g.mol⁻¹, Aldrich), benzylamine (Aldrich), and dichloromethane (Fischer, >99%) were used as received. Hostasol (Thioxantheno[2,1,9-dej]isochromene-1,3-dione) was synthesized as described in Limer, A. J.; Rullay, A. K.; San Miguel, V.; Peinado, C.; Kelly, S.; Fitzpatrick, E.; Carrington, S. D.; Brayden, D.; Haddleton, D. M. React. Funct. Polym. 2006, 66, 51-64). N-hydroxysuccinimide-2-bromopropionate was synthesized as previously reported (Lecolley, F.; Tao, L.; Mantovani, G.; Durkin, I.; Lautru, S.; Haddleton, D. M. Chem. Commun. 2004, 2026-2027).

### Polymerization Procedure

For a 12 KDA copolymer, the procedure is the following. A dry Schlenk tube was charged with Cu(I) Br (0.179 g, 1.25 mmol), N-succinimidyl-2-bromopropionate (0.31 g, 1.25 mmol), PEGMA₄₇₅ (4.75 g, 10.0 mmol) and hostasol methacrylate monomer (0.09 g, 0.19 mmol, 1.6 mol. % with respect to PEGMA₄₇₅), toluene (15.0 g, 0.16 mol) and a magnetic follower. The mixture was then subjected to five freeze-pump-thaw degassing cycles. Degazed N-(ethyl)-2-pyridylmethanimine (0.39 ml, 2.76 mmol) was added and the resulting brown solution was stirred at 50° C. (time zero of the polymerization) for 48 h. Samples were removed periodically using degassed syringes and quenched in liquid nitrogen for conversion and molecular weight analysis (samples for molecular weight analysis were prepared by passing over an acidic alumina column to remove the copper complexes). The final polymer was passed over acidic alumina column, precipitated in petroleum ether to removed unreacted hostasol methacrylate, dialyzed against slightly acidic water and lyophilized.

For the 45 KDA copolymer, same materials and experimental procedures were used except that monomer over initiator molar ratio was 31.8.

Conjugation of NHS-PEG-Functionalised Polymers with Fluorescent Marker to Hair in Diglyme

In a 100 mL cylindrical flask, 10 g (Mₙ=11 800 g/mol) of PEGMA/Hostasol NHS-ester chain-end copolymer (reactive NHS-copolymer) was introduced followed by 70 ml of diglyme (diethylene glycol dimethyl ether) and 3.48 g of anhydrous triethylamine. 10 g of bleached hair switches were then introduced. The reaction medium was shaken slowly during for 30 minutes at room temperature, to ensure complete reaction. The hair switches were taken out of the reaction vessel and were washed repeatedly with deionised water. After the washing procedure was complete, the hair switch was naturally dried overnight before analysis.

A deep fluorescence especially when the focus is made at the top of the treated hair fibres is apparent. Theses results demonstrate that conjugation to the hair fibre has been successful.

Conjugation of NHS-PEG-Functionalised Polymers with Fluorescent Marker to Hair in IPA/Water Mixtures

In a 100 mL cylindrical flask, 10 g (Mₙ=11 800 g/mol) of PEGMA/Hostasol NHS-ester chain-end copolymer (reactive NHS-copolymer) was introduced followed by 70 ml of a mixture of isopropanol/water (95/5; vol./vol.) and 3.48 g of anhydrous triethylamine. 10 g of bleached hair switches were then introduced. The reaction medium was shaken slowly during for 30 minutes at room temperature, to ensure complete reaction. The hair switches were taken out of the reaction vessel and were washed repeatedly with deionised water. After the washing procedure was complete, the hair switch was naturally dried overnight before analysis.

A deep fluorescence especially when the focus is made at the top of the treated hair fibres is apparent. Theses results demonstrate that conjugation to the hair fibre has been successful.

### DSC Studies

### Sample Preparation

European hair fibres were bleached two times with L'Oreal platifiz precision powder and Oxydant crème (1:1.5) for 30 min, rinsed completely with running water from the tap, and naturally dried overnight before the next application. These hair fibres were immersed in 1% active solution and water (as a control) at pH 5.5 for 1 h respectively, rinsed with distilled water for 30 sec and naturally dried overnight. And then they were cut into ˜2 mm length with scissors.

### DSC Method

About 6 mg of sample was weighted into a pressure resistant (25 bar), stainless steel, large volume pan (60 μl capacity). 50 μl of water was added and the pan was sealed. Samples were then mixed using a rotary mixer and left overnight to allow the water to equilibrate throughout the sample. Samples were run through a temperature programme of 120-180° C. at a rate of 5° C./min in 30 ml/min nitrogen atmosphere (FIG. 3). The helix transition temperature was collected and analyzed with one-way ANOVA. Each sample was carried out at least four times.

### DSC Data

The DSC data of pre-washed and post-washed bleached hair fibre as well as bleached and PEGylated hair fibres is given in Table 1 below. A high denaturation temperature (Td) indicates that the hair is not denatured,

TABLE 1
DSC data of bleached and modified bleached hair fibres

| PEGylation | | | | | |
|---|---|---|---|---|---|
| study | | Td (° C.) | | | Td (° C.) |
| 12 KDA | 1 | 151.98 | 45 KDA | 1 | 151.85 |
| PEGylated | | | PEGylated | | |
| hair | | | hair | | |
| Prewash | 2 | 151.53 | Prewash | 2 | 152.45 |
| | 3 | 151.57 | | 3 | 152.44 |
| | 4 | 152.65 | | 4 | 153.09 |
| | 5 | 149.60 | | 5 | 149.88 |
| | Mean | 151.47 | | Mean | 151.94 |
| | Std. | 1.14 | | Std. | 1.23 |
| | dev. | | | dev. | |
| 12 KDA | 1 | 153.32 | 45 KDA | 1 | 153.43 |
| PEGylated | | | PEGylated | | |
| hair | | | hair | | |
| Postwash | 2 | 153.84 | Postwash | 2 | 151.91 |
| | 3 | 152.94 | | 3 | |
| | 4 | 152.72 | | 4 | |
| | 5 | 153.36 | | 5 | |
| | Mean | 153.24 | | Mean | 152.67 |
| | Std. | 0.43 | | Std. | 1.07 |
| | dev. | | | dev. | |
| Control 2 bleached hair | | | | | |
| | 1 | 146.72 | | 1 | 146.72 |
| | 2 | 144.27 | | 2 | 144.27 |
| | 3 | 142.64 | | 3 | 142.64 |
| | 4 | 143.81 | | 4 | 143.81 |
| | 5 | 143.57 | | 5 | 143.57 |
| | Mean | 144.20 | | Mean | 144.20 |
| | Std. | 1.53 | | Std. | 1.53 |
| | dev. | | | dev. | |

Td = Denaturation temperature ° C.

The DSC data clearly show that the denaturation temperatures of postwashed bleached hair fibre PEGylated with PEG of molecular weights 12 KD has been increased by 9° C. and the bleached hair modified with 45 KD has been increased by 7.74° C. compared to bleached hair fibre. This implies that PEGylation significantly enhances the properties of damaged hair and can be employed for repairing damaged hair fibre.

1. A method of protecting hair by covalently bonding to the hair a polymeric compound comprising polyethylene glycol.

2. A method according to claim 1 wherein the polymeric compound comprises at least one polymeric segment of polyethylene glycol covalently attached to a central backbone.

3. A method according to claim 1 in which the central backbone comprises a acrylate, methacrylate group or mixtures thereof.

4. A method according to claim 1 in which the molecular weight Mw of the polyethylene glycol or polyethylene glycol segment is from 5,000 to 60,000.

5. A method according to claim 3 in which the number of acrylate, methacrylate groups within the central back bone is from 2 to 10.

6. Use of an reactive derivative of polyethylene glycol for derivatising hair

7. Use according to claim 6 in which the reactive derivative is selected from acrylated polyethylene glycol reactive or methacrylated polyethylene polyethylene glycol or mixtures thereof.

8. Use according to claim 6 in which the reactive group of the reactive derivative of polyethylene glycol is selected from the group consisting of is α-aldehyde, α-maleimide, α-N-hydroxy succinimide, or α-azlactone.

9. Use according to claim 8 in which the reactive group is α-N-hydroxy succinimide

10. A hair fibre covalently bonded to a polymeric compound comprising polyethylene glycol.

11. A hair fibre according to claim 10 in which the polymeric compound comprising polyethylene glycol. Is acrylated polyethylene glycol or a methacrylated polyethylene glycol or mixtures thereof.
